(19) United States
(12) Patent Application Publication
(10) Pub. No.: US 2015/0223669 A1
(43) Pub. Date: Aug. 13, 2015

(54) MULTI-MODAL MEDICAL EXAMINING INSTRUMENT AND RELATED METHOD FOR ENABLING MULTIPLE EXAMINATION MODES

(71) Applicant: Welch Allyn, Inc., Skaneateles Falls, NY (US)

(72) Inventor: Ervin Goldfain, Syracuse, NY (US)

(21) Appl. No.: 14/622,010

(22) Filed: Feb. 13, 2015

Related U.S. Application Data
(60) Provisional application No. 61/939,400, filed on Feb. 13, 2014.

(51) Int. Cl. A61B 1/00; A61B 3/14; A61B 5/00; A61B 1/05; A61B 1/227; A61B 1/303; A61B 3/12; A61B 3/00

(52) U.S. Cl. CPC A61B 1/0019; A61B 3/12; A61B 3/14; A61B 3/0041; A61B 1/05; A61B 1/227; A61B 1/303; A61B 5/0077; G02B 3/14

(57) ABSTRACT

A medical diagnostic instrument or optical module includes a housing retaining an optical assembly having at least one variable focus lens and an electronic imaging element each disposed along a common optical axis. The electronic imaging element is axially movable along the imaging axis to provide focusing along with the at least one variable focus lens with regard to a target of interest in order to enable multiple and disparate examination modes to be conducted.

### CROSS REFERENCE TO RELATED APPLICATION

This application claims priority to U.S. Application Ser. No. 61/939,400, filed Feb. 13, 2014 under relevant portions of 35 U.S.C. §119. The entire contents of this document are herein incorporated by reference.

### TECHNICAL FIELD

This application is generally related to the field of diagnostic medicine and more specifically to a medical diagnostic instrument and/or instrument module defining a singular optical architecture to enable multiple examination modes of various targets and over an extended range of working distances in order to permit expanded functionality.

### BACKGROUND

Several different types of medical apparatus are individually used to examine a patient, such as those which are used by a physician or other caregiver during a typical wellness visit. For example, an otoscope can be used for examining the outer ear, an ophthalmoscope can be used to conduct an examination of the eyes, and a colposcope can be used for examining the cervix of a female patient. Each of these instruments are equipped for either visual examination by a caregiver or can include an electronic imaging element that permits viewing of the target on a computer monitor or a dedicated display. In each of the foregoing examinations, the working distance (i.e., the distance between the field of view and the distalmost optical component (i.e., the component closest to the target)) varies greatly and therefore dedicated optical systems are required.

It is an ongoing and general need in the medical field to be able to incorporate multiple examination functions into a single medical diagnostic instrument. Prior attempts have been made, such as described in Applicant's prior U.S. Pat. No. 6,106,457, in which various instrument heads (i.e., ophthalmoscope, otoscope, skin microscope) are separately and individually attached to a single instrument body (i.e., a handheld digital camera). Each of the instrument heads according to this reference is configured with a unique optical assembly that cooperates with that of the digital camera when an instrument head intended for a specific type of examination is attached. It would be more advantageous to enable the examination of different areas of a patient to be performed using a single medical diagnostic instrument, while still maintaining a singular optical architecture.

### BRIEF DESCRIPTION

Therefore and according to a first aspect, there is provided a medical diagnostic instrument comprising a housing that retains an optical assembly, the optical assembly comprising a variable focus lens assembly, such as a so-called “liquid lens” or a focus tunable lens, that is aligned along an optical or imaging axis with an electronic imaging element. At least one focusing mechanism permits the electronic imaging element to be moved along the imaging axis and further enables adjustment of the variable focus or tunable lens assembly with regard to an intended target of interest of a patient.

Advantageously, the herein described instrument is configured to enable the performance of multiple examination modes with regard to a patient, each examination mode requiring a different working distance between the imaging assembly and the target of interest (e.g., ear, eye, face). Preferably, the instrument can adaptably capture still or live images over a range of working distances between about 10 mm and 400 mm, depending on the intended target and examination to be conducted. According to one version, a single instrument can be configured to selectively perform multiple and disparate examination modes without significant modification or requiring the addition of optical elements.

According to one version, the optical assembly further includes a wide angle lens element proximally disposed from a target of interest and distally disposed relative to the variable focus lens assembly along the imaging axis. In one version, the electronic imaging element can be part of a digital camera or other “smart” device (e.g., a smartphone, a tablet) having a contained electronic imaging element, wherein either element or the device retaining the electronic imaging element is selectively movable along the optical axis of the instrument in order to provide gross or initial focusing of a target of interest and wherein the variable focus lens assembly can be selectively adjusted to provide fine focusing of the intended target, the focusing operations can be performed manually using a mechanism provided on the device or using automatic controls or software.

As noted above, the herein described instrument can be used over a range of working distances depending on the intended target of the patient to be examined. In an exemplary version, a medical diagnostic instrument can be configured to operate selectively over a working distance of about 20 mm for purposes of using the instrument as a skin microscope, a working distance of about 75 mm to enable use of the instrument as a facial camera and about 300 mm or more to enable use of the instrument in a colposcopic examination mode. Other suitable examination modes can be contemplated that similarly employ this developed optical architecture.

In addition and according to at least one version an illumination assembly, such as an array of LEDs (e.g., white LEDs) or other source of light sufficient to conduct an examination, can be further provided for illuminating a target of interest either through direct or indirect illumination relative to the imaging axis of the instrument. According to at least one version, the output of the illumination assembly can also be adjusted, as needed. In at least one version, the illumination assembly can further include at least one filtering element, again depending on the examination conducted (e.g., a green filter for vaginal examinations).

In terms of use, the herein described instrument can examine multiple different areas of a patient. For example and according to a first examination mode, a portion of the skin of the patient such as a wart, mole or other form of anomaly can be examined. In a second examination mode, the face of the patient and/or the caregiver can be examined, such as for identification or for other diagnostic purposes. According to yet another version, the instrument can be further used in a colposcopic mode, each of the above examination modes requiring different working distances and in which each examination mode can be successfully performed using the same optical and illumination assembly. In at least one of the herein described modes, examination can be enhanced, if needed, using a zoom or a magnification feature.

According to another aspect, there is provided a method for enabling multiple modes for examining a patient over a range of working distances and magnifications, the method comprising providing an optical assembly, including a variable focus lens assembly and an electronic imaging element each disposed along an imaging axis, configuring the electronic imaging element proximal to the variable focus lens assembly, and enabling the electronic imaging element to be movable along the imaging axis over a fixed range such that the electronic imaging element and variable focus lens each can provide focusing capability with regard to a target of interest.

In at least one version, a wide-angle optical element is disposed along the imaging axis distal of the variable focus lens assembly. According to this method, multiple examination modes can be enabled, each examination mode requiring a different working distance. In at least one version, the electronic imaging element can be moved axially along the imaging axis using a manual or automatic focusing feature. In at least one version, the electronic imaging element can be retained within an enclosure that is connected to a focusing member (e.g., a focusing knob) or other actuable element, such as provided on the instrument housing, to enable axial movement of the electronic imaging element along the imaging axis. A display can be connected to the electronic imaging element through a hard-wired or wireless means in order to view the intended target.

According to one version, the attached electronic imaging element can be moved linearly to provide gross focusing capability, while the variable focus lens assembly can then be adjusted to suitably provide finer focusing of a captured still or live image of the intended target.

According to yet another aspect, there is provided an adaptive module for at least one medical diagnostic instrument to extend the range of effective working distances of the instrument, the imaging module comprising a module housing retaining an optical assembly. The optical assembly comprises a variable focus lens that is aligned along an imaging axis with an electronic imaging element and a mechanism for separately and axially and independently moving the electronic imaging element along the imaging axis and also adjusting the variable focus lens with regard to a target of interest.

According to yet another aspect, there is provided a medical diagnostic instrument comprising a housing having an optical assembly including at least one variable focus lens, and a device having an embedded electronic imaging element such as a smartphone or tablet computer. An attachment mechanism is configured for engaging the housing with the smart device, wherein the coupling mechanism substantially aligns the variable focus lens and the electronic imaging element along a common optical axis. A mechanism can be further provided for separably and axially moving the electronic imaging element along an imaging axis in order to provide a first level of focusing and for independently adjusting the variable focus lens to provide a second level of focus with regard to an intended target of interest.

One advantage provided by the herein described medical diagnostic instrument is greater functionality using a device having a single optical architecture to perform a plurality of disparate medical examination procedures over a varied range of potential working distances and magnifications.

Another advantage provided is that of reduced inventory requirements for a medical environment, such as a health care facility or a primary physician's office.

Yet another advantage provided that enablement between examination modes that are defined by significantly varied working distances are easily realized without significant modification and using a single optical architecture.

These and other features and advantages will be readily apparent with reference to the following Detailed Description, which should be read in conjunction with the accompanying drawings.

### BRIEF DESCRIPTION OF THE DRAWINGS

FIG. 3 is a perspective view of a known diagnostic instrument system which includes various instrument heads separately attachable to a single instrument housing, the system being capable of performing various examination modes;

FIG. 2 is a schematic view of a diagnostic instrument made in accordance with an exemplary embodiment;

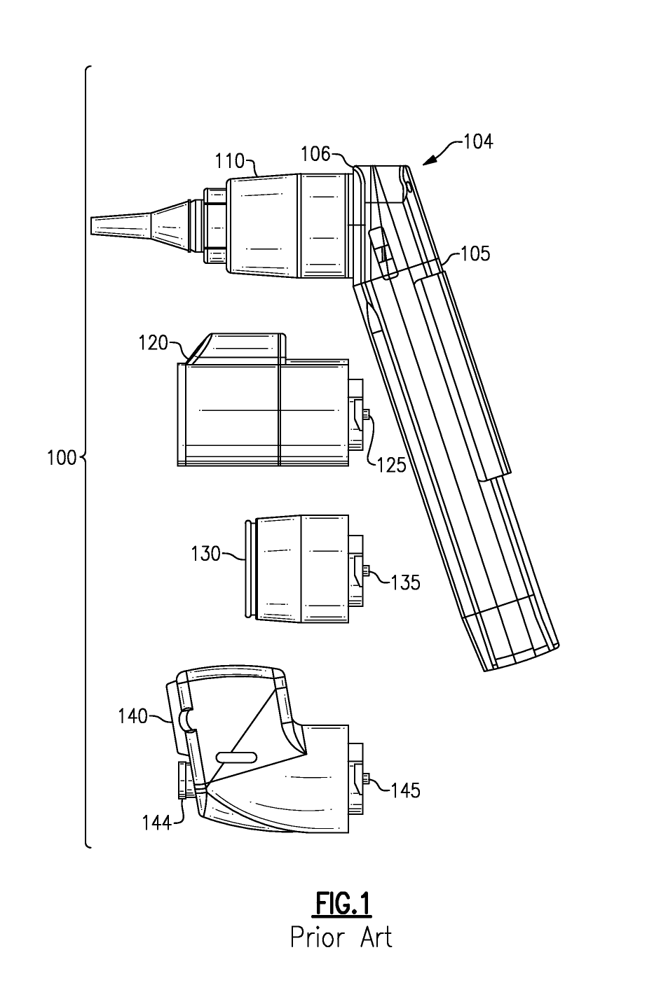

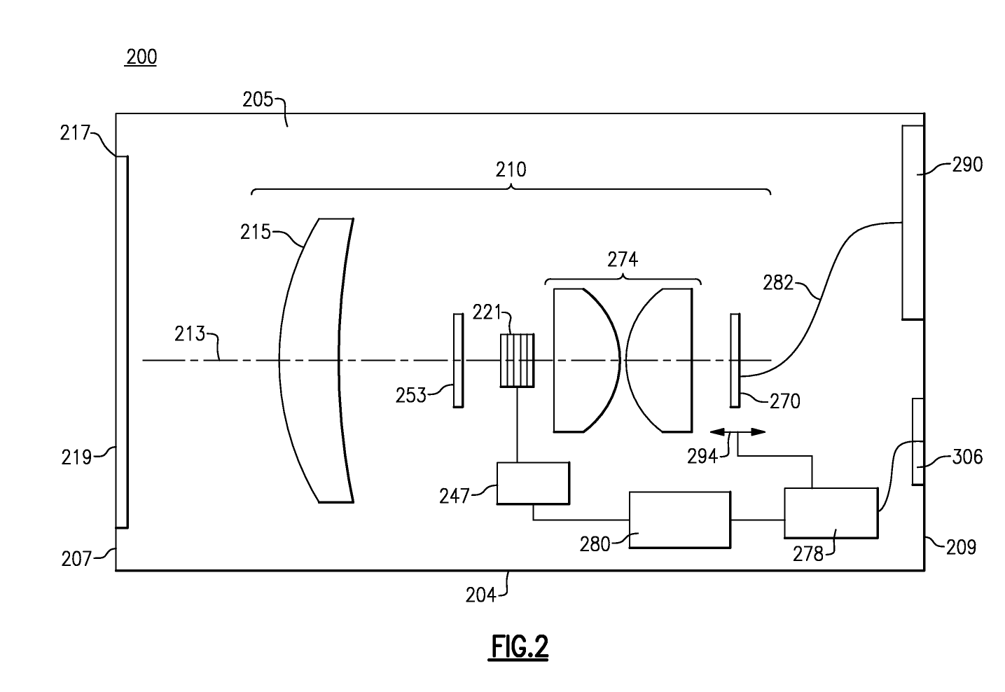

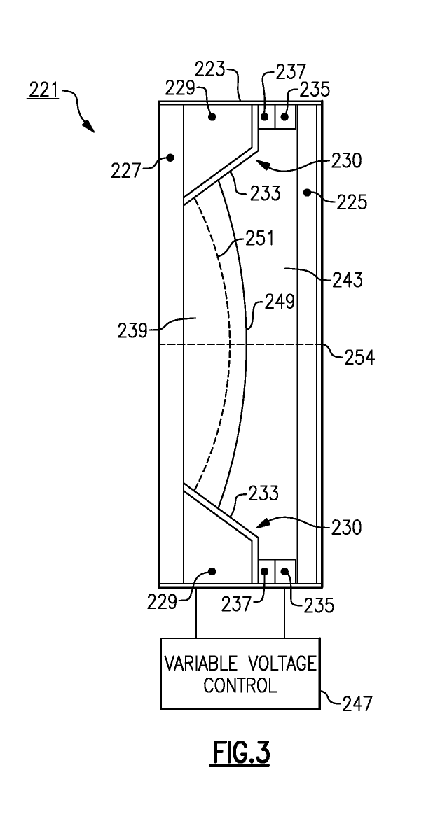

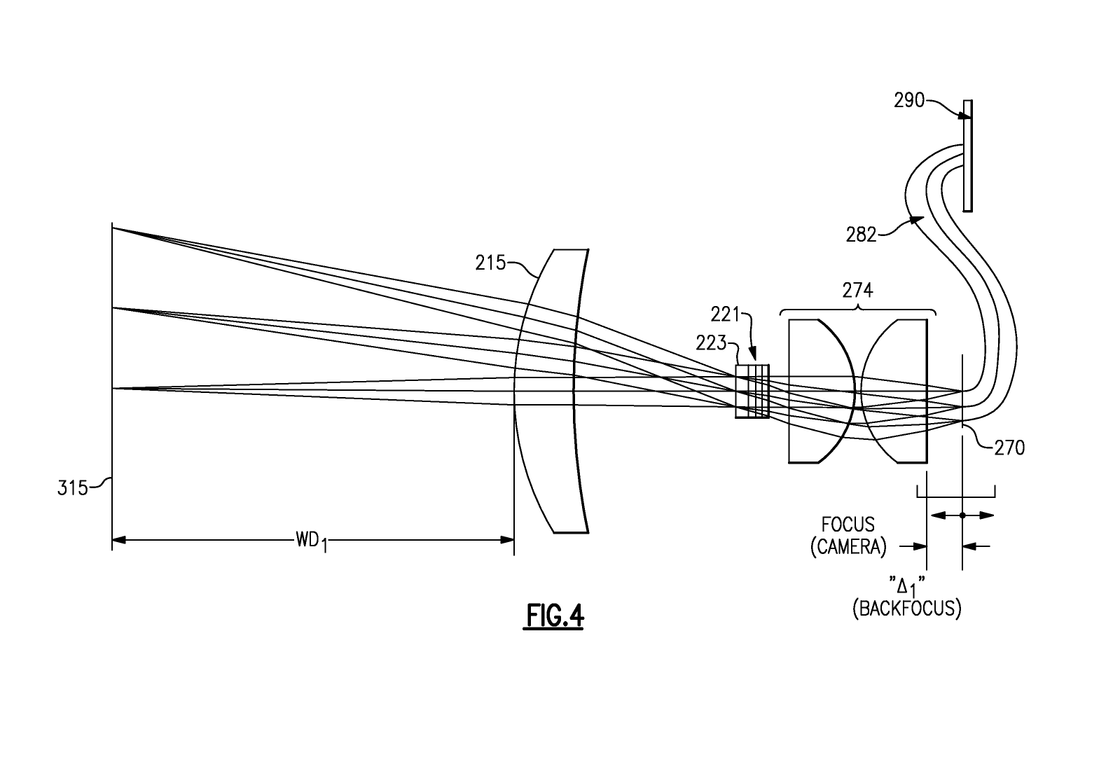

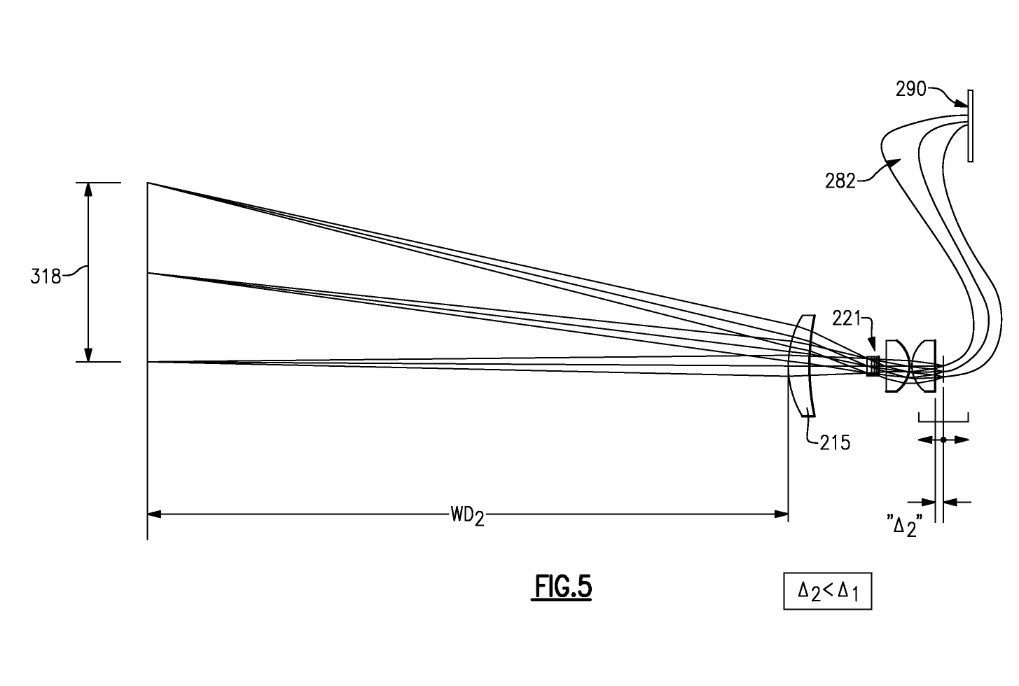

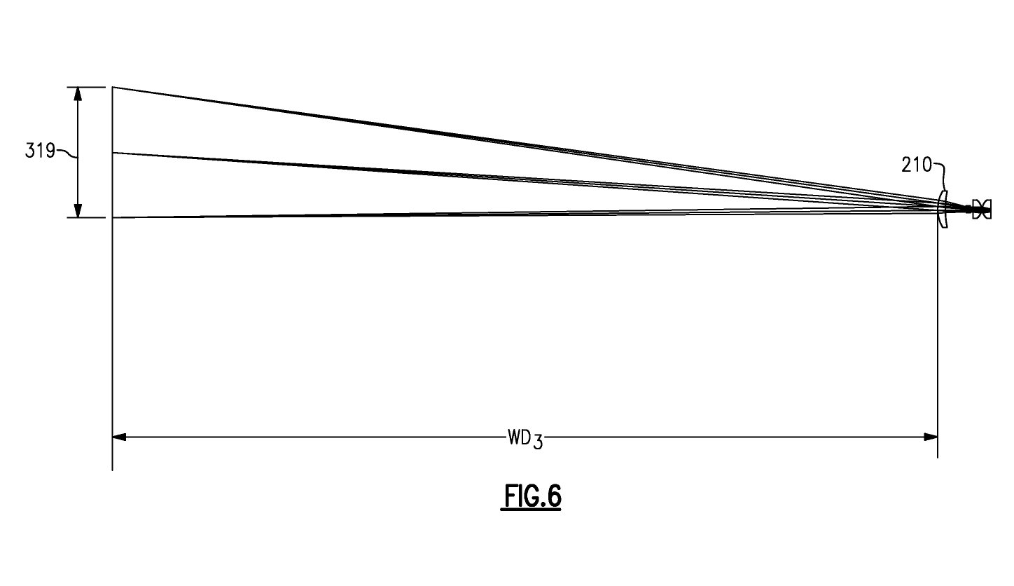

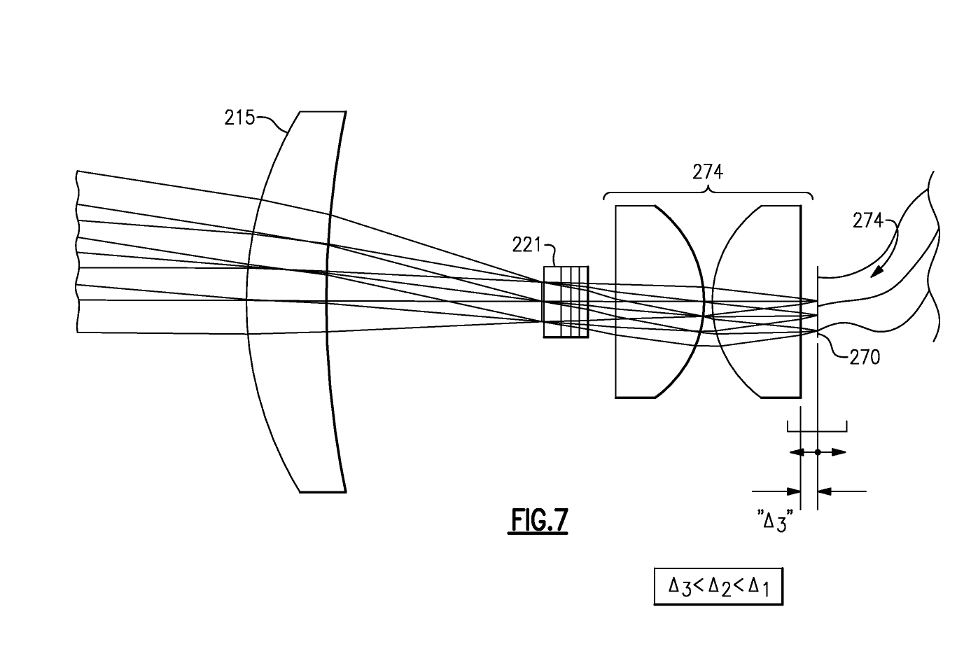

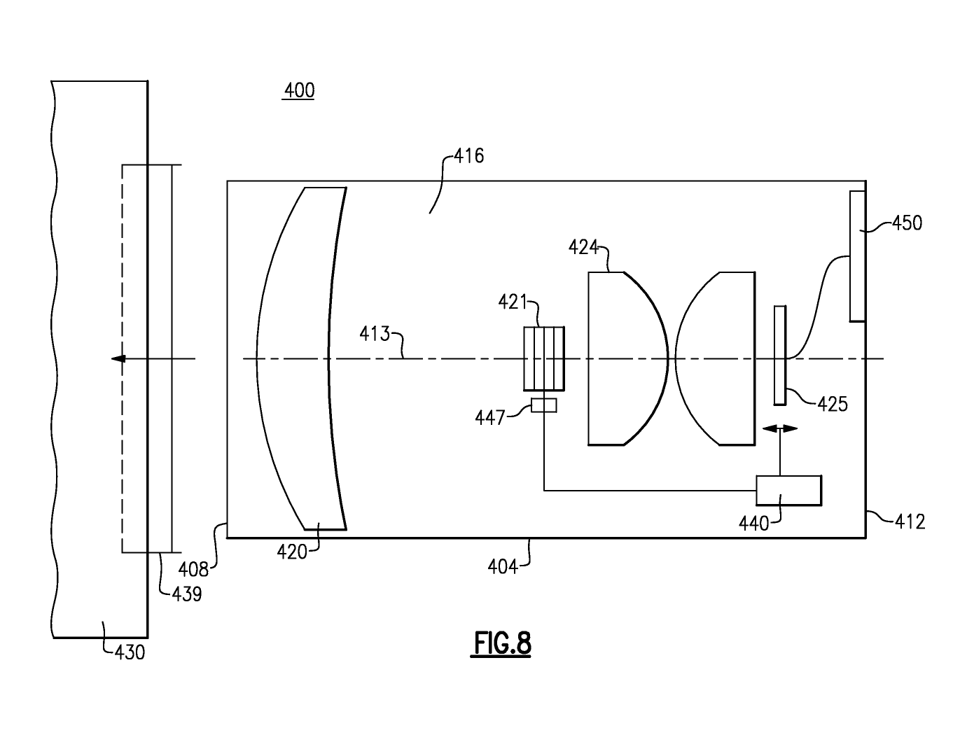

FIG. 3 illustrates an exemplary variable focusing lens assembly for use in the diagnostic instrument of FIG. 2;

FIG. 4 is a schematic view of the diagnostic instrument of FIG. 2, and configured according to a first examination mode;

FIG. 5 is a schematic view of the instrument of FIG. 2, and configured according to a second examination mode;

FIG. 6 is a schematic view of the instrument of FIG. 2, and configured according to a third examination mode;

FIG. 7 is an enlarged view of a portion of the diagnostic instrument of FIG. 6; and

FIG. 8 is a schematic view of an adaptive module that includes a singular exemplary optical architecture for examining a patient.

### DETAILED DESCRIPTION

The following description relates to a medical diagnostic instrument made in accordance with exemplary embodiments that is operable in at least two examination modes, each examination mode requiring a different range of working distances in regard to an intended target of a patient, as well as an adaptive module that is configured for use with at least one diagnostic instrument. The examination modes that are described herein are exemplary and merely intended to demonstrate the inventive concepts. As a result, it will be readily apparent to those of sufficient skill that other suitable versions could be contemplated that embody the concepts described herein for different medical targets of interest. In addition and in the course of discussion, various terms are used in order to provide a suitable frame of reference with regard to the accompanying drawings. These terms which may include “distal”, “proximal”, “upper”, “lower”, “top” and “bottom, among others, should not be viewed as limiting of the present invention, except where so specifically indicated.

In addition, the accompanying drawings are intended to convey the salient features, but should not be interpreted as providing scaling that should be relied upon with regard to size and geometry.

For purposes of background, reference is made to FIG. 1 that illustrates a prior art medical diagnostic instrument system 100. The diagnostic instrument system 100 includes a series of instrument heads iso, 120, 130 and 140, each of which are separately and releasably couplable to the distal end 106 of a digital camera 104. This diagnostic instrument system 100 is described in greater detail in U.S. Pat. No. 6,106,457, the entire contents of which are incorporated herein by reference. Each of the instrument heads 110, 120, 130 and 140 include a separate optical assembly contained therein that is unique to a form of examination being performed and the working distance required in regard to a target of interest, when each instrument head is releasably and separably coupled to the retained electronic imager (not shown) of the digital camera 104.

In use, each instrument head 110, 120, 130, 140 is separately and releasably attached to the distal end 106 of the camera housing 105, using complementary bayonet features (features 125, 135 and 145 being shown at a proximal end of each instrument head 120, 130, 140) or other convenient attachment mechanism in which an attached otoscopic instrument head 110 is aligned with the digital camera 104 and then twisted to position the desired instrument head in a predetermined position and orientation when attached to the distal end 106 of the digital camera housing 105 to enable various types of examination to be performed, as needed. Each instrument head 110, 120, 130, 140, when attached, is further optically coupled via the contained optics that directs an image of the target of interest to the contained electronic imager and creates a dedicated diagnostic instrument that includes the camera housing 105 and the elected instrument head 210. As described in greater detail by the above-incorporated patent, an illumination assembly 144 can be further provided within at least one of the instrument heads 140 for dispersing sufficient light at the target of interest for viewing by a caregiver. As previously noted, teardown of an instrument head and assembly of a different instrument head is required prior to each examination procedure that is to be conducted.

Referring to FIG. 2, there is shown a medical diagnostic instrument 200 which is made in accordance with an exemplary embodiment. The diagnostic instrument 200 is defined by a housing 204 (partially shown) that is further defined by a distal end 207 and an opposing proximal end 209, as well as a substantially hollow interior 205 that is sized to retain a plurality of components as herein described, including an optical or imaging assembly 210 and an electronic imaging element 270.

The imaging assembly 210 according to this embodiment includes a plurality of optical components, each being disposed along a common optical or imaging axis 213. More specifically, a distalmost optical element, e.g., a wide angle lens 215, is disposed in fixed relation adjacent the distal end 207 of the housing 204. The distal end 207 of the housing 204 includes an opening 217 sized to permit incoming and outgoing light to pass therethrough. A cover glass or other optical transparent member 219 can be fitted to the opening 217 to prevent the ingress of contaminants, such as dirt and dust. A variable focus lens assembly 221, such as, for example, a so-called “liquid lens”, is disposed proximally relative to the wide angle lens 215 and aligned along the optical axis 213.

Referring to FIG. 3, an exemplary variable focus lens assembly 221 is herein described for purposes of this embodiment. This assembly 221 includes a housing 223 that incorporates a pair of parallel transparent windows 225, 227, a first electrode 229 having a frusta-conical opening 230, an insulating layer 233 disposed on the first electrode 229, a second electrode 235, an insulator 237, a drop of insulating liquid 239 located on the conical insulating layer 233 and on the window 227, and an electrically conductive liquid 243 filling the remainder of the housing 223. The filled conductive liquid 243 is in electrical contact with the second electrode 235, while the insulating liquid 239 and the conductive liquid 243 are in contact along a meniscus region represented by a solid line 249. The insulating liquid 239 and conductive liquid 243 are both transparent, are immiscible, have different optical indices, and have substantially the same density. The conductive liquid 243 can, for example, be water mixed with salts and the insulative liquid 239 can be, for example, oil. In one embodiment, the lens assembly can include one or more electrically controllable variable focus liquid lenses. While the depicted variable focus lens assembly 221 includes a single variable focus liquid lens this assembly could alternatively be configured, for example, with first and second variable focus lens the assembly having a controllable variable iris (not shown) located between the lenses. The variable iris controls the amount of light passing through the liquid lens assembly comprising the multiple liquid lenses. Examples of the foregoing and additional details concerning the exemplary liquid lens assembly are provided in WO 2013/071153 and U.S. Pat. No. 7,553,020 B2, the entire contents of each document herein incorporated by reference.

In operation and when no voltage is applied, the exemplary system is at rest. In this configuration, the drop of insulating liquid 239 naturally takes the shape of the solid line 249, which is designated by a reference curve. An axis 254 is perpendicular to the transparent windows 225, 227 and passes through the center of the meniscus (reference) curve 249. This latter axis 254 constitutes the optical axis of the variable lens assembly 221, which when assembled is made coincident with the optical axis 213.

Applying a non-zero voltage V from a variable voltage control 247 shown schematically between the first electrode 229 and the second electrode 235 creates an electrical field localized in the region surrounding the electrodes 229, 235. As a consequence, the conductive liquid 243 deforms the insulating liquid drop 239 and the reference curve resultantly assumes the shape designated by the dashed line 251. This results in a variation of the focal length of the liquid lens assembly 221. A range of applied voltages will result in a range of various radii of curvature for the dashed line 251 and therefore a corresponding range of optical powers and focal lengths for the liquid lens assembly 221.

According to at least one version at an instrument interface, the liquid lens assembly 221 can be switched between various focal lengths. Additional details regarding this component are described in previously cross-referenced WO2013/071153 and U.S. Pat. No. 7,553,020 B2. According to this embodiment, an aperture stop 253, FIG. 2, can be disposed distally of the variable focus lens assembly 221 in order to prevent stray light from being transmitted to the electronic imaging element 270, as discussed herein.

Other forms of variable focusing lens assemblies can be substituted for use herein. For example, so-called “focus tunable” lenses made by Optotune AG consist of shape-changing lenses whose variable operation is based upon a combination of a thin polymeric membrane and contained optical fluids. More specifically, a container is filled with an optical fluid that is sealed with the polymeric membrane. In one version, a circular ring is configured to apply pressure to the center of the membrane to selectively shape the lens using electrical current to cause the membrane to deflect and change the radius of the lens. This can be done by causing the circular ring to be pushed toward the membrane or by exertion of pressure to an outer portion of the membrane or by pumping liquid into and out of the container. For example, an Optotune EL-10-30 lens relies upon electrical current to create pressure to change (shape) the lens. Such versions are described in U.S. Pat. No. 8,000,022 and U.S. Patent Application Publication No. 2013/0114148, each of these documents being incorporated herein by reference.

Referring back to FIG. 2 and proximally of the variable focus lens assembly 221 is a pair or doublet of optical lenses 274, also arranged along the imaging axis 213 of the instrument 200. The electronic imaging element 270, such as a CCD or one made from a CMOS architecture, is further disposed proximally of the doublet 274. This element 270 includes a plurality of pixels that are capable of creating a still or live image. The imaging element is preferably supported for axial movement. One support/enclosure for an electronic imaging element is described in U.S. Pat. No. 8,469,882, the entire contents of which are herein incorporated by reference. The electronic imaging element 270 is electrically connected to a circuit board (not shown) that is further connected to a motor assembly, shown schematically as 278, that is configured to move the support containing the electronic imaging element 270, each of the motor assembly 278, and the variable voltage control 247 being connected to a controller or processor 280. Alternatively, the electronic imaging element 270 can be moved using a focusing knob or other member (not shown) provided on the instrument housing 204 that cooperates with at least one cam or element (not shown) that produces axial (translational) movement of the electronic imaging element, such as described, for example, in the cross-referenced U.S. Pat. No. 8,469,882. In addition and according to this exemplary embodiment, the electronic imaging element 270 is tethered by a flexible cable 282 to a liquid crystal display 290 and is enabled for axial movement, as shown by arrows 294, along the optical axis 213 over a predetermined linear range using the motor assembly 278. The controller 280 can be caused to operate each of the motor assembly 278 and the variable voltage assembly 247 using at least one switch or other actuable element 306 that can be provided, for example, on the exterior of the instrument housing 204. Alternatively, this control can be made through a wireless connection to a remote control (not shown) using infrared, Bluetooth, WiFi, or other type of connection. Though the electronic imaging element is shown as integrated within the instrument housing according to this exemplary embodiment, a separate device having an embedded electronic element, such as a smartphone or a tablet computer, can also be configured for attachment to the proximal end of the instrument housing 204. The proximal end of the housing 204 can be configured for engaging the separate device, such that when coupled the variable focus lens assembly and the electronic imaging element are substantially aligned along the common optical axis 213. In this latter version, the controls provided on the separate device can be used to provide a first level of focusing wherein independent adjustments of the variable focus lens enable or provide a second level of focus with regard to an intended target of interest.

Referring to FIGS. 4-7, various examination modes of the herein described instrument 200 are described that can be easily accomplished using the contained imaging assembly 210, each of these examination modes requiring a significantly different working distance in regard to the intended target. According to a first examination mode and as shown in FIG. 4, skin microscopy is enabled wherein the instrument 200 can be used to detect under magnification, various skin defects such as scars, moles, cuts and the like. For this specific examination mode, the working distance (WD₁) between the wide angle lens 215 at the distal end 207, FIG. 2, of the instrument housing 204, FIG. 2, and the target of interest (i.e., skin) is approximately 20 mm in which the field of view (FOV) 315 is approximately 16 mm. To effect focusing, the electronic imaging element 270 is axially movable using the motor assembly 278 as controlled by the actuable buttons 306 at the proximal end of the instrument housing 204 along the imaging axis 213 over a range of Δ₁ relative to the doublet 274 and the remainder of the optical assembly. According to this embodiment, Δ₁ is about 3 to 8 mm. Fine focusing can then be provided, as needed, using the variable voltage control 247 to adjust the focal length of the variable lens assembly 221. The resulting image can be viewed on the attached display 290, which according to this embodiment is attached to the proximal end 209, FIG. 2, of the instrument housing 204, FIG. 2, According to this embodiment, a zoom or magnification mode can be further provided through the controller 280 to enable enhanced viewing of the area of interest or the image can be rotated.

Referring to FIG. 5, a second examination mode is schematically illustrated for the herein described instrument 200 and more specifically relates to a facial camera mode. This latter examination mode can be used, for example, for verifying proper identification of either the patient or the caregiver. According to this version, the working distance (WD₂) between the wide angle lens 215 at the distal end of the instrument and the target of interest is approximately 75 mm and the field of view (FOV), half of which is shown by arrow 328, is approximately 50 mm. As in the prior described mode, the electronic imaging element 270 is configured for axial movement (Δ₂) along the imaging axis 213 in order to enable rough focusing to be done wherein the contained variable focus lens assembly 221 can then be adjusted for fine focusing of the resulting image using the variable voltage control 247. According to this mode, Δ₂ is about 3-8 mm.

Referring to FIGS. 6 and 7, a third exemplary examination mode is shown schematically. According to this exemplary embodiment, a colposcopic mode can be enabled that is also commonly executed by the herein described instrument 200. In this specific mode, the working distance (WD₃) between the distal most optical element of the system (the wide-angle lens 215) and the target of interest (e.g., the cervix) is over 310 mm, which is significantly greater than the working distance (WD₁, WD₂) of each of the prior examination modes discussed herein. The field of view (FOV), half of which is shown by arrow 319, for this mode is approximately 100 mm. As in the prior examination modes, focusing is done using the electronic imaging element 27 initially over an axial range (Δ₃) with fine focusing being done through adjustment of the variable focus lens assembly 221 with respect to the target of interest. According to this specific mode, Δ₃ is approximately 2.5-6 mm.

In addition and according to at least one version an illumination assembly, such as an array of LEDs (e.g., white or multispectral LEDs) or other source of light sufficient to conduct an examination (not shown), can be further provided for illuminating a target of interest either through direct or indirect illumination relative to the imaging axis 215 of the instrument 200. According to at least one version, the output of the illumination assembly can also be adjusted, as needed. In at least one version, at least one filtering element or spectral illumination can also be included, again depending on the examination conducted (e.g., a green filter or illumination for colposcopic applications). This filtering can be done using an optical element or through software, while any multispectral illumination can be accomplished by tuning a multispectral LED.

With reference to FIG. 8, there is provided an adaptive optical module 400 that can be attached to at least one diagnostic instrument 430, shown partially. The herein described optical module 400 is defined by a module housing 404 having a distal end 408 and an opposing proximal end 412, including an interior 416 that is appropriately sized to accommodate a plurality of optical components. More specifically and according to this exemplary embodiment, the herein described adaptive optical module 400 is configured with the prior discussed optical architecture including a variable focus lens assembly 421, such as a liquid lens assembly, FIG. 3, which is disposed proximally from a wide angle lens 420 disposed at the distal end 408 of the module housing 404 along a defined optical or imaging axis 413. An electronic imager assembly 425 is also aligned along the common optical axis 413 and is disposed proximal to the variable focus lens assembly 421 and an intermediately disposed optical doublet 424. The electronic imager assembly 425 is supported for movement along the optical axis using a controller 440 that is also connected to a variable voltage control 447 used to adjust the variable focus lens assembly 421 and is additionally connected to a display 450, provided on the housing 416 or separately attached thereto using either a hard-wired or wireless (e.g., Bluetooth) connection.

The module housing 404 is further provided with at least one attachment feature or means (not shown) that enables the adaptive optical module 400 to be releasably attached to at least one or interchangeable with a plurality of instrument housings, such as instrument 430 (partially shown). The module can be fitted within the interior of the instrument housing, or attached to the proximal end thereof. According to this version, the adaptive module 400 is configured to fit within a ring 439 disposed at the proximal end of the instrument 430, the module housing 404 being sized for accommodation in a releasable manner such that the housing 404, when attached, is aligned with the optical axis 413 of the instrument 430. Other suitable mounting configurations could also be utilized.

### PARTS LIST FOR FIGS. 1-8

100 diagnostic medical instrument
104 hand held digital camera
105 housing, camera
106 distal end, camera
110 instrument head, otoscopic
120 instrument head, surface microscope
125 proximal end
130 instrument head, general view
135 proximal end
140 instrument head, magnifying
145 proximal end
344 adjustable lens assembly
200 diagnostic medical instrument
204 housing
205 hollow interior
207 distal end
209 proximal end
210 optical or imaging assembly
213 imaging axis
215 wide angle lens
217 opening
219 cover glass
221 variable focus lens (liquid lens) assembly
223 housing
225 transparent window
227 transparent window
229 first electrode
230 frusto-conical opening
233 insulating layer
235 second electrode
237 insulator
239 drop of insulating liquid
243 electrically conductive liquid
247 variable voltage control
249 meniscus region
251 curve
253 aperture stop
254 axis
270 electronic imaging element
274 doublet
278 motor assembly
280 controller or processor
282 flexible cable
290 display
294 arrows
306 actuable element
315 field of view
318 field of view
319 field of view
400 optical module
404 module housing
408 distal end
412 proximal end
413 optical or imaging axis
420 wide angle lens
421 variable focus lens assembly
424 doublet
425 electronic imaging element
430 instrument
439 proximal ring
440 controller
447 variable voltage control
WD₁ working distance
WD₂ working distance
WD₃ working distance
Δ₁ axial movement range
Δ₂ axial movement range
Δ₃ axial movement range

It will be readily apparent that other variations and modifications will be contemplated using the inventive concepts as discussed, and according to the following claims.

1. A medical diagnostic instrument comprising:
a housing;
an optical assembly including:
at least one variable focus lens assembly; and
an electronic imaging element, each of the variable focus lens assembly and the electronic imaging element being disposed along a common optical axis; and
a mechanism for separably and axially moving the electronic imaging element along the imaging axis to provide a first level of focusing and for independently adjusting the variable focus lens assembly to provide a second level of focus with regard to an intended target of interest.

2. The diagnostic instrument as recited in claim 1, wherein the at least one variable focus lens assembly is defined by a liquid lens.

3. The diagnostic instrument as recited in claim 1, wherein the at least one variable focus lens assembly is defined by a focus tunable lens.

4. The diagnostic instrument as recited in claim 1, wherein the diagnostic instrument is configured to perform multiple examination modes based on selective focusing of at least one of the electronic imaging element and the at least one variable focus lens.

5. The diagnostic instrument as recited in claim 4, wherein the instrument is configured to perform at least two different examination modes, each examination mode having a different working distance between a distalmost optical element of the optical assembly and the intended target of interest.

6. The diagnostic instrument as recited in claim 1, further comprising a display connected to the electronic imaging element.

7. The diagnostic instrument as recited in claim 1, further comprising a mechanism that is programmed to automatically move the electronic imaging element along the imaging axis to provide coarse focusing and adjust the variable focus lens to provide fine focusing with regard to a target of interest.

8. The diagnostic instrument as recited in claim 1, including a mechanism connected to the electronic imaging element for moving the electronic imaging element selectively along the imaging axis.

9. A method for enabling multiple examination modes of a patient using a single diagnostic instrument, the method comprising:
providing an optical assembly including at least one variable focus lens assembly and an electronic imaging element commonly disposed along an optical axis;
configuring the electronic imaging element proximal to the variable focus lens assembly; and
enabling the electronic imaging element for movement along the optical axis over a fixed linear range such that each of the electronic imaging element and at least one variable focus lens assembly can provide focusing capability with regard to a target of interest.

10. The method as recited in claim 9, including the step of providing a wide-angle lens distal of the variable focus lens assembly along the optical axis, the wide-angle lens enabling a field of view for separate examination modes.

11. The method as recited in claim 9, including the step of linearly enabling movement of the digital imaging element to provide coarse focusing and then adjustment of the variable focus lens assembly to enable fine focusing.

12. The method as recited in claim 9, wherein the adjustment of the electronic imaging element and the at least one variable focus lens assembly is performed automatically.

13. The method as recited in claim 9, wherein the adjustment of the electronic imaging element and the at least one variable focus lens assembly is performed manually.

14. The method as recited in claim 10, including enabling the diagnostic instrument to transmit focused images of a target of interest over at least two examination modes, each examination mode having a different working distance.

15. An imaging module for use with at least one medical diagnostic instrument, the module comprising:
a module housing having an interior;
an electronic imaging element disposed within said module housing;
at least one variable focus lens assembly aligned with the electronic imaging element along an imaging axis; and
a mechanism for linearly moving the electronic imaging element along the imaging axis to provide a first level of focus and for adjusting the variable focus lens assembly to provide a second level of focus relative to a target of interest.

16. The module of claim 15, wherein the at least one variable focus lens assembly is defined by a liquid lens.

17. The module of claim 15, wherein the at least one variable focus lens assembly is defined by a focus tunable lens.

18. The module of claim 15, further comprising a wide-angle lens disposed distally of the variable focus lens assembly.

19. The module of claim 15, including at least one feature enabling attachment to a medical diagnostic instrument.

20. The module of claim 19, wherein the at least one feature permits releasable securement to at least one medical diagnostic instrument.
